(19) United States
(12) Patent Application Publication
(10) Pub. No.: US 2025/0312378 A1
(43) Pub. Date: Oct. 9, 2025

(54) COMPOSITION COMPRISING ANTI-FOLIC ACID AGENT FOR DIFFERENTIATION OF MESENCHYMAL STEM CELL INTO CARTILAGE

(71) Applicant: EWHA UNIVERSITY-INDUSTRY COLLABORATION FOUNDATION, Seoul (KR)

(72) Inventors: Byeongmoon JEONG, Seoul (KR); Ye Jin WOO, Yongin-si, Gyeonggi-do (KR)

(21) Appl. No.: 18/692,563

(22) PCT Filed: Sep. 16, 2022
(86) PCT No.: PCT/KR2022/013880

§ 371 Date: Nov. 5, 2024

(30) Foreign Application Priority Data
Sep. 17, 2021 (KR) 10-2021-0124425

(51) Int. Cl. A61K 35/28; A61K 9/00; A61K 9/06; A61K 31/145; A61K 31/505; A61K 31/519; A61K 47/34

(52) U.S. Cl. CPC A61K 35/28; A61K 9/0024; A61K 9/06; A61K 31/145; A61K 31/505; A61K 31/519; A61K 47/34

(57) ABSTRACT

Embodiments relate to a composition for inducing differentiation of stem cells into chondrocytes, a pharmaceutical composition for treatment of cartilage injury diseases, and a sustained-release dosage form for inducing differentiation of stem cells into chondrocytes, wherein the compositions and dosage form each comprise an antifolate. Using the composition for inducing differentiation of stem cells into chondrocytes according to one embodiment, it is possible to selectively induce stem cells to differentiate into chondrocytes without inducing differentiation into other cell types.

### TECHNICAL FIELD

The present invention relates to a composition for inducing differentiation of stem cells into chondrocytes containing an antifolate and a method of treating cartilage injury disease using the same, and is capable of differentiating stem cells into chondrocytes economically, stably, and efficiently by using a clinically approved antifolate such as dapsone, pralatrexate, trimethoprim, or a combination thereof.

### BACKGROUND ART

Osteoarthritis is a degenerative joint disorder resulting in destruction of articular cartilage. Because cartilage has no blood vessels, normal tissue repair mechanisms are not applied thereto. In addition, because cartilage tissue has a low cell density, it is difficult to self-regenerate properly from local cells. Currently, a method of transplanting mature autologous chondrocytes is used as a cell-based therapy for osteoarthritis. However, differentiated chondrocytes have limited proliferative capacity, and thus it can be a major challenge to obtain an adequate number of cells required for transplantation. In addition, patients with osteoarthritis are usually elderly, and there is a problem in that the proliferation level of autologous cartilage cells decreases as the patient ages. Therefore, stem cell therapy has been actively studied in the field of regenerative medicine as a treatment method for osteoarthritis that can overcome these limitations. Stem cells have much better proliferation and regeneration capacities than mature somatic cells, including chondrocytes. In regenerative medicine, it is an important prerequisite for therapeutic applications of stem cells to control the fate and behavior of stem cells and induce stem cells to differentiate into specific cell types. The differentiation and functional activity of stem cells are generally regulated by a mixture of growth factors and signaling molecules. Naturally or chemically synthesized compounds have advantages over high-molecular-weight growth factors such as proteins in that they are accurate, efficient, reversible, and economical as means for differentiating stem cells. However, compounds that can induce stem cells to differentiate into chondrocytes are known to be very limited, such as TD-198946, a thienoindazol derivative, prostaglandin E2, dexamethasone, kartogenin (KGN), and oxopiperazine derivatives (5{i,2}).

Meanwhile, antifolates are a class of antimetabolite medications that antagonize the actions of folic acid. Antifolates inhibit cell division, DNA/RNA synthesis and repair, and protein synthesis. Therefore, antifolates are used as antibiotics and cytotoxic substances to treat cancer, autoimmune diseases, psoriasis, and bacterial infections. Antifolates clinically approved by the U.S. Food and Drug Administration (FDA) include dapsone, methotrexate, pemetrexed, pralatrexate, proguanil, pyrimethamine, and trimethoprim.

Folic acid is known to significantly improve chondrogenic differentiation of mesenchymal stem cells. Korean Patent No. 10-2197871 discloses a method of differentiating stem cells into chondrocytes using folic acid. Although the above patent document mentions that antifolates may be used for purposes similar to folic acid, it does not actually show that stem cells can be differentiated into chondrocytes using antifolates, which are used as anticancer agents, unlike folic acid.

### DISCLOSURE

### Technical Problem

According to one embodiment of the present invention, provided is a composition for inducing differentiation of stem cells into chondrocytes, containing an antifolate.

According to another embodiment of the present invention, provided is a pharmaceutical composition for treating cartilage injury disease, containing an antifolate and stem cells.

According to still another embodiment of the present invention, provided is a sustained-release dosage form for inducing differentiation of stem cells into chondrocytes, including the composition for inducing differentiation of stem cells into chondrocytes according to one embodiment.

### Technical Solution

Throughout the present specification, it is to be understood that when any part is referred to as “including” any component, it does not exclude other components, but may further include other components, unless otherwise specified.

Throughout the specification, words of degree, such as “about”, “substantially”, and the like are used herein in the sense of “at, or nearly at, when given the manufacturing and material tolerances inherent in the stated circumstances” and are used to prevent the unscrupulous infringer from unfairly taking the stated disclosure where exact or absolute figures are stated as an aid to understanding the present application.

As used throughout the present specification, the term “step of doing . . . ” or “step of . . . ” does not mean “step for doing . . . .”

Throughout the present specification, the term “combination(s) thereof” included in the expression of the Markush form means one or more mixtures or combinations selected from the group consisting of the components described in the Markush form representation, and includes one or more selected from the group consisting of the above components.

Throughout the present specification, the expression “A and/or B” means “A or B” or “A and B”.

Throughout the present specification, “differentiation” refers to a phenomenon in which cells become more specialized in structure or function during growth after division and proliferation thereof, that is, cells, tissues, etc. of an organism change in their shape or function to perform the given task.

Throughout the present specification, the term “chondrocytes” may include chondrocytes induced to differentiate from stem cells, or cells differentiating into chondrocytes.

Throughout the present specification, the term “medium” refers to a mixture for in vitro culture or differentiation of cells such as stem cells, which contains components essential for growth and proliferation of the cells, such as sugar, amino acids, various nutrients, serum, growth factors, minerals, and the like.

Hereinafter, embodiments and examples of the present application will be described in detail with reference to the accompanying drawings. However, the present application may not be limited to these embodiments and examples and the accompanying drawings.

A composition for inducing differentiation of stem cells into chondrocytes according to one embodiment of the present invention contains an antifolate.

The composition is able to amplify the expression of genes or proteins contained in the chondrocytes differentiated from stem cells. For example, the genes or proteins may be type II collagen (COL II), SRY-box 9 (SRY-box transcription factor 9, SOX 9), aggrecan (ACAN), core-binding factor β subunit (CBFβ), runt-related transcription factor 1 (RUNX1), or combinations thereof, without being limited thereto.

The concentration of the antifolate may be in the range of 0.01 μM to 100 μM, 0.01 to 10 μM, 0.05 μM to 0.5 μM, 0.05 μM to 0.2 μM, or 0.05 μM to 0.15 μM. If the concentration of the antifolate is lower than the lower limit of the above range, the induction of differentiation of stem cells into chondrocytes may not occur, and if the concentration of the antifolate is higher than the upper limit of the above range, the efficiency of induction may be lowered due to cytotoxicity.

The antifolate may be any clinically used drug. For example, the antifolate may be dapsone, methotrexate, pemetrexed, pralatrexate, proguanil, pyrimethamine, trimethoprim, or a combination thereof. Preferably, the antifolate may be dapsone, pralatrexate, trimethoprim, or a combination thereof. More preferably, the antifolate may be pralatrexate.

The stem cells may be selected from the group consisting of adipose-derived mesenchymal stem cells, bone marrow-derived mesenchymal stem cells, umbilical cord blood-derived stem cells, embryonic stem cells, tonsil-derived mesenchymal stem cells, and synovial mesenchymal stem cells, without being limited thereto.

The mesenchymal stem cells may be selected from the group consisting of bone marrow-derived stem cells, adipose-derived stem cells, tonsil-derived stem cells, synovial stem cells, and combinations thereof, without being limited thereto. The mesenchymal stem cells may be obtained from bone marrow, tissue, embryo, umbilical cord blood, blood, or body fluid, without being limited thereto.

In one embodiment, the mesenchymal stem cells may be tonsil-derived mesenchymal stem cells, specifically, tonsil-derived mesenchymal stem cells (TMSCs). TMSCs have several advantages in that they have the potential for differentiation into multiple lineages and against immunosuppression, similar to other MSCs, may be easily obtained from tonsil tissue discarded after tonsillectomy, and cause less serious age-related complications and have a faster proliferation rate than bone marrow-derived MSCs (BMSCs) and adipose-derived MSCs (AMSCs).

The composition for inducing differentiation of stem cells into chondrocytes may be used by treating a stem cell culture medium therewith. The medium may include any medium that is generally used for culturing of stem cells. For example, the medium may be DMEM, MEM, BME, RPMI 1640, F-10, F-12, DMEM-F12, α-MEM, G-MEM, MSCGM, IMDM, MacCoy's 5A, AmnioMax, AminoMaxII complete Medium, or Chang's Medium MesemCult-XFMedium, without being limited thereto.

A pharmaceutical composition for treating cartilage injury disease according to another embodiment of the present invention contains an antifolate and stem cells.

The cartilage injury disease may be arthritis, cartilage injury, cartilage defect, degenerative arthritis, rheumatoid arthritis, fracture, plantar fasciitis, lateral humerus epicondylitis, osteomalacia, or a combination thereof, without being limited thereto.

The pharmaceutical composition is able to promote the regeneration of cartilage tissue in the joint by inducing specific differentiation of endogenous stem cells or transplanted exogenous stem cells into chondrocytes.

The pharmaceutical composition may be injected directly into a patient's joint according to a known method, or may be transplanted together with a scaffold after three-dimensional culture, without being limited thereto. For example, when the pharmaceutical composition is to be injected into a patient, the dosage of injection thereof may be adjusted in consideration of several related factors such as the disease to be treated, the severity of the disease, the route of administration, and the patient's weight, age and sex.

The pharmaceutical composition may further contain a pharmaceutically acceptable carrier that is used for injection or transplantation into a patient. The carrier may vary depending on the mode of administration.

In addition, the pharmaceutical composition may further contain other pharmaceutically acceptable ingredients to promote regeneration of cartilage tissue, including, but not limited to, glucosamine, chondroitin, collagen, hyaluronic acid, minerals such as calcium and magnesium, vitamins such as vitamin D and vitamin C, and the like.

According to still another embodiment of the present invention, provided is a sustained-release dosage form for inducing differentiation of stem cells into chondrocytes, including the composition for inducing differentiation of stem cells into chondrocytes.

The sustained-release dosage form may be in the form of a thermogel. The term “thermogel” refers to a material which, after injected into a target area in a solution state, undergoes a sol-to-gel transition in response to heat to form a gel.

When the sustained-release dosage form is in a gel form, it may be suitable for application to a three-dimensional culture method. The term “three-dimensional culture” refers to a culture method in which cells grow in a cluster so as to form a three-dimensional shape, for example, a sphere or ellipsoid, not a conventional cell monolayer culture method in which cells are cultured so that a cell monolayer is formed by cell growth. Preferably, pellet culture may be used. For example, the pellet culture is effective in maintaining the chondrocyte phenotype, and may provide an extracellular environment similar to that found in the initial cartilage tissue generation by inducing cell-cell adhesion through easy aggregation of cells by centrifugation.

The sustained-release dosage form may include a thermogel and stem cells. When stem cells are suspended in an aqueous polymer solution at a temperature equal to or lower than the sol-gel transition temperature and injected, the stem cells may be incorporated in the hydrogel formed in situ during the heat-induced sol-gel transition, and nutrients and differentiation factors may be incorporated in the pores of the hydrogel, so that the stem cells may be differentiated into chondrocytes more efficiently and stably and, at the same time, the differentiated chondrocytes may be released slowly into cartilage tissue or the nutrients and differentiation factors may be released slowly from the hydrogel, thus inducing endogenous stem cells also to differentiate into chondrocytes.

The sustained-release dosage form may include a poly(ethylene glycol)-poly(L-alanine) copolymer. The copolymer may have a molecular weight of 1,000 Da to 10,000 Da, 1,000 Da to 5,000 Da, or 1,000 Da to 2,000 Da. The polydispersity index of the copolymer may be 0.5 to 1.5, 0.8 to 1.5, or 1.0 to 1.2. The critical micelle concentration (CMC) of the copolymer may be 0.01 wt % to 1.0 wt %, 0.01 wt % to 0.1, or 0.01 wt % to 0.05 wt %.

The sustained-release dosage form may be prepared by mixing an antifolate and stem cells in an aqueous solution of poly(ethylene glycol)-poly(L-alanine) at a sol temperature and heating the mixture to a gelation temperature to form a gel. The aqueous solution of poly(ethylene glycol)-poly(L-alanine) may be used mixed with a medium, and may be used in an amount of 5 w/w % to 20 w/w %, 5 w/w % to 15 w/w, or 8 w/w % to 12 w/w %, based on the weight of the medium. The stem cells contained in the sustained-release dosage form may be at passage 3 to passage 10, passage 5 to passage 8, or passage 7, and the number thereof may be 0.01×10⁶ to 1×10⁶ cells, 0.1×10⁶ to 10×10⁶ cells, or 0.3×10⁶ to 0.8×10⁶ cells.

The sol temperature may be 10° C. to 25° C., 15° C. to 25° C., or 18° C. to 22° C., and the gelation temperature may be 5° C. to 40° C., or 35° C. to 38° C.

### Advantageous Effects

Using the composition for inducing differentiation of stem cells into chondrocytes according to one embodiment of the present invention, it is possible to selectively induce stem cells to differentiate into chondrocytes without inducing differentiation into other cell types. The antifolate that is used in the present invention is a clinically approved drug and has advantages in that it may be applied both in vivo and ex vivo because it is safe for the human body, and has excellent reproducibility, productivity, and economic efficiency.

### BRIEF DESCRIPTION OF DRAWINGS

FIG. 1A shows the ¹H-NMR spectra of PEG-PA and PEG (CF₃COOD), FIG. 1B shows the FTIR spectrum of PEG-PA (10.0 wt % in D₂O), FIG. 1C shows the CD spectra as a function of polymer concentration in aqueous PEG-PA solution, and FIG. 1D shows the shift of the CD band as a function of polymer concentration.

FIG. 2A shows the phase diagram of an aqueous PEG-PA block copolymer solution, FIG. 2B shows the modulus of the aqueous PEG-PA block copolymer solution (10.0 wt %) as a function of temperature, and FIG. 2C shows a field emission-scanning electron microscopy image of a thermogel produced from the aqueous PEG-PA block copolymer solution (10.0 wt %) at 37° C. (scale bar: 10 μm).

FIG. 3 shows the release profile of each candidate compound from a thermogel produced from an aqueous PEG-PA solution.

FIG. 4A is a graph showing the results of evaluating the cytotoxicity of a candidate material for chondrogenic differentiation of TMSCs using a PEG-PA thermogel containing an antifolate, and FIG. 4B is a graph showing the mRNA expression level of COL II, a chondrogenic biomarker, by a candidate material under 3D culture conditions.

FIG. 5 is an image showing dead and live cells in a PEG-PA 3D matrix containing an antifolate. Here, 1, 4, 7, and KGN represent dapsone, pralatrexate, tramethoprim, and KGN, respectively, and the scale bar is 50 μm.

FIG. 6A shows the mRNA expression level of COL II in a PEG-PA 3D matrix containing each antifolate, FIG. 6B shows the mRNA expression level of SOX 9 in the matrix, FIG. 6C shows the mRNA expression level of ACAN in the matrix, and FIG. 6D shows the mRNA expression level of COL I in the matrix. Here, 1, 4, 7, and KGN represent dapsone, pralatrexate, trimethoprim, and KGN, respectively.

FIG. 7A shows the protein expression level of COL II in a PEG-PA 3D matrix containing each antifolate, FIG. 7B shows the protein expression level of SOX 9 in the matrix, FIG. 7C shows the protein expression level of ACAN in the matrix, and FIG. 7D shows the protein expression level of COL I in the matrix. DAPI and PHD stain the nucleus blue and the cytosol green. The scale bar is 25 μm. Here, 1, 4, 7, and KGN represent dapsone, pralatrexate, trimethoprim, and KGN, respectively.

FIG. 8A is a Western blot image showing the nuclear translocation of CBFβ in TMSCs, FIG. 8B is a graph showing the band intensity in the nucleus versus the cytosol in Western blot analysis, and FIG. 8C shows immunofluorescence images of CBFβ. The scale bar is 25 μm. Here, 1, 4, 7, and KGN represent dapsone, pralatrexate, trimethoprim, and KGN, respectively.

FIG. 9 depicts immunofluorescence images showing the expression of RUNX1 in TMSCs (scale bar: 25 μm). Here, 1, 4, 7, and KGN represent dapsone, pralatrexate, trimethoprim, and KGN, respectively.

### BEST MODE

The present invention relates to a composition for inducing differentiation of stem cells into chondrocytes, containing an antifolate.

### MODE FOR INVENTION

Hereinafter, the present invention will be described in more detail by way of the following examples. However, the following examples are intended only to illustrate the present invention and are not construed to limit the scope of the present invention in any way.

### Example 1. Production and Characterization of Thermogel

### 1-1. Production of Thermogel

A poly(ethylene glycol)-poly(L-alanine) (PEG-PA) block copolymer was prepared as a thermogel. Specifically, a polymer was synthesized by ring-opening polymerization of a PEG-PA monomer with N-carboxy anhydrides of L-alanine (NCA-A; Onsolution, Korea) using α-amino-ω-methoxy poly(ethylene glycol) (Mn=2,000 Daltons; Creative PEGWorks, USA) as an initiator, as disclosed previously (Kim, H. et al., Biomacromolecules 2020, 21, 3176-3185). The synthesized polymer was purified by fractional precipitation with diethyl ether, and the residual solvent was evaporated under vacuum. Then, the purified polymer was dialyzed in deionized water using a membrane with a cutoff molecular weight of 1,000 Da and then lyophilized.

### 1-2. Analysis of Structure and Polydispersity Index of Thermogel

To analyze the molecular weight of PEG-PA, gel permeation chromatography (SP930D; Younglin, Korea) using N,N-dimethylformamide as an eluent and H-NMR spectrometry using trifluoroacetic acid-D (CF₃COOD) (300 MHz NMR spectrometer, Bruker, USA) were performed. In addition, the PEG-PA copolymer was suspended in water at 25° C. and the secondary structure thereof was determined by FT-IR spectroscopy (Nicolet iS10, Thermo Scientific, USA) and circular dichroism (CD) spectroscopy (J-1500, JASCO, Japan). The critical micelle concentration of PEG-PA was investigated by CD spectroscopy in the polymer concentration range of 0.01 to 1.0 wt %.

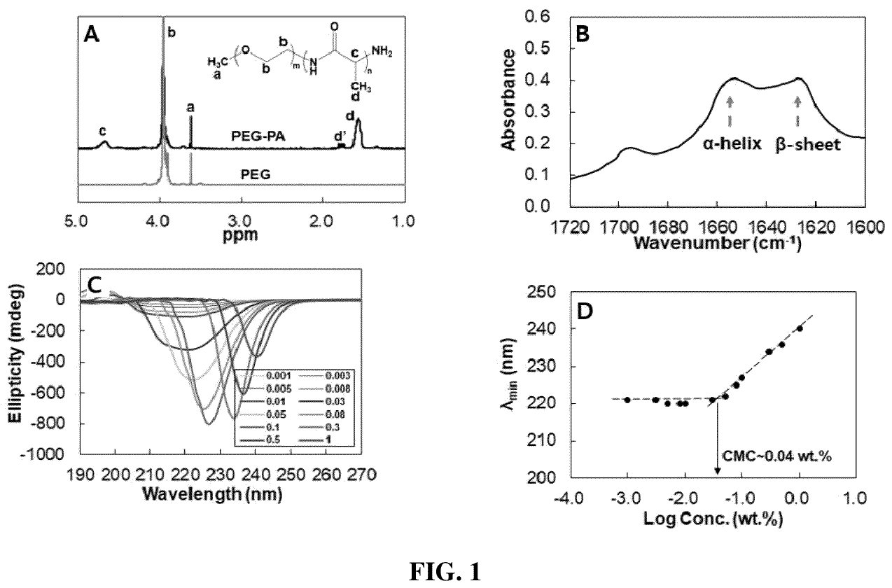

The molecular weight of the PEG-PA block copolymer was calculated to be 2,000 to 1,000 Da by comparing the area of the ethylene glycol peak (—CH₂CH₂O—) at 3.8 to 4.1 ppm and the methyl peak area of alanine (—NHCH(CH₃)CO—) at 1.4 to 1.7 ppm in the ¹H-NMR spectrum (FIG. 1A). The polydispersity index of the PEG-PA block copolymer was 1.1 as measured by gel permeation chromatography.

The secondary structure of the PEG-PA self-assembly at a concentration of 10.0 wt % in D₂O at 25° C. was examined by the FT-IR spectrum (FIG. 1B). The peaks at 1,625 cm⁻¹ and 1,652 cm⁻¹ in the FTIR spectrum were assigned to β-sheet and α-helix structures, respectively. The circular dichroism spectrum of the aqueous PEG-PA solution was obtained as a function of concentration. At a concentration of 0.01 wt %, the negative band with a minimum at 220 nm indicates a β-sheet structure, while the negative band at 208 nm indicates an α-helix structure. The CD spectrum showed negative bands at 208 and 220 nm, and as the polymer concentration increased from 0.001 wt % to 1.0 wt %, the band red-shifted to 230-240 nm, indicating that self-assembly of PEG-PA occurred due to the amphiphilic nature of the polymer composed of a hydrophobic PA block and a hydrophilic PEG block (FIG. 1C). The intersection of two lines extrapolated from the CD spectrum indicates self-assembly (micelles) of the block copolymer (FIG. 1D). The critical micelle concentration (CMC) of the PEG-PA block copolymer was 0.04 wt %.

### 1-3. Investigation of Sol-Gel Transition of Thermogel

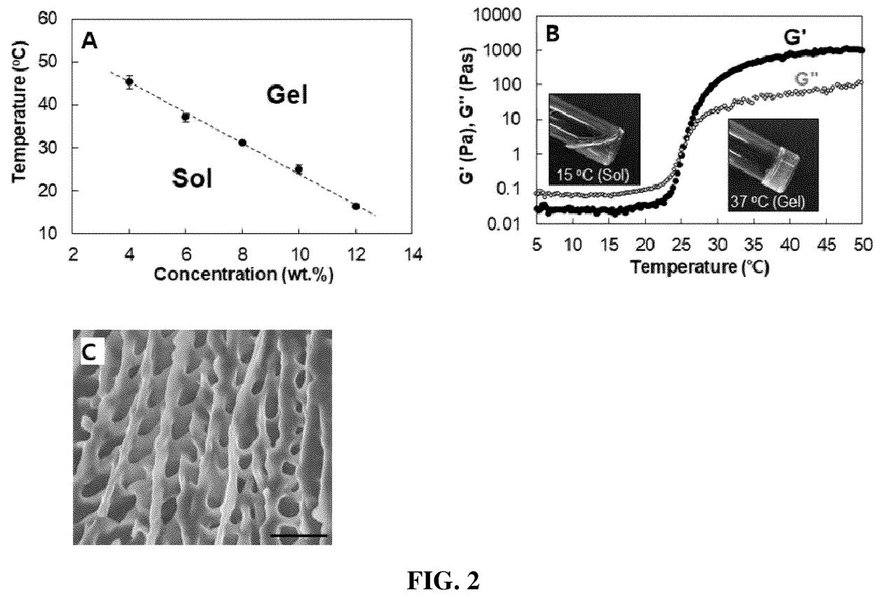

To investigate the sol-gel transition of the aqueous PEG-PA block copolymer solution as a function of the polymer concentration, a phase diagram was obtained using the test-tube inverting method. Specifically, the solution (1.0 mL) was placed in a test tube having an inner diameter of 11 mm, and the transition point at which the solution changes from a fluid state (sol) to a non-fluid state (gel) was determined while the temperature was increased by 1.0° C./step. Each data point was determined as the mean of three measurements. As the temperature increased, the aqueous PEG-PA block copolymer solution transitioned from a sol to a gel in the polymer concentration range of 4.0 to 12.0 wt %. As the polymer concentration increased from 4.0 to 12.0 wt %, the transition temperature decreased from 45° C. to 16° C. (FIG. 2A). The aqueous polymer solution was in a flowable sol state at concentrations below 4.0 wt %, whereas it was in a gel state at a concentration equal to or higher than 12.0 wt. % in the experimental temperature range of 10° C. to 60° C.

The modulus of the aqueous polymer solution (10.0 wt %) was investigated as a function of temperature (FIG. 2B). The change in modulus of the aqueous polymer solution (10.0 wt. %) was investigated as a function of temperature (5 to 50° C.) using a rotational rheometer (Bohlin Gemini 150, Malvern, England). The solution was placed between two parallel plates having a diameter of 20 mm and a gap of 0.5 mm, and the modulus of the solution was recorded under controlled stress (0.4 Pa) at a frequency of 1.0 rad/s and a heating rate of 1.0° C./min. The storage modulus (G′) intersects the loss modulus (G″) at 25° C., and G′ and G″ represent the elasticity and viscosity magnitudes of the complex modulus, respectively. When G′ is greater than G″, the elasticity overwhelms the viscosity, indicating a sol-gel-transition.

The aqueous PEG-PA block copolymer solution (10.0 wt %) used in the 3D culture study of TMSCs showed a gel state with a G′ value of 600 Pa at 37° C., indicating strong enough properties to retain the cells. At 15° C., the aqueous copolymer solution became a low-viscosity sol state with a G′ value lower than 0.03 Pa. FIG. 2B shows the sol and gel state images of the aqueous polymer aqueous.

### 1-4. Observation of Porosity of Thermogel

To observe the porosity of the gel, a field emission-scanning electron microscopy (FE-SEM) image of the gel was acquired. Specifically, the PEG-PA block copolymer thermogel (10.0 wt %) at 37° C. was quenched in liquid nitrogen (−196° C.), freeze-dried, and observed with a field emission scanning electron microscope (JSM-6700F, JEOL, Japan). Because the polymer gel has a porous structure, it can immobilize and sustainably release stem cells during 3D cell culture and transport nutrients and metabolites (FIG. 2C).

### Example 2. Determination of Drug Release Profile of Thermogel

The drug release profile of the thermogel was determined in vitro. Specifically, dapsone, pralatrexate, trimethoprim, or KGN (0.02 mg) was dissolved in the aqueous PEG-PA block copolymer solution (10.0 wt %, 0.2 mL). The temperature of the polymer solution containing the drug was increased to 37° C. to cause a sol-gel transition. After 30 minutes, phosphate-buffered saline (PBS) (1.2 mL, 37° C.) was added thereto, and the mixture was placed in a shaking thermostatic bath with 40 strokes/min at 37° C. PBS was replaced at specific sampling intervals. The drug released into the medium, PBS, was analyzed by a high-performance liquid chromatography system (HPLC; Waters 1525B, USA) equipped with a photodiode detector (Waters 2998, USA) and a Jupiter@5 μm C18 300A LC column (Phenomenex, USA). As a mobile phase, a mixed solvent of methanol/water (40/60 v/v) containing trifluoroacetic acid (0.1%) was used for dapsone, pralatrexate and trimethoprim, and a mixed solvent of acetonitrile/water (35/65 v/v) containing formic acid (0.1%) was used for KGN. The flow rate was set at 1.0 ml/min. The amount of drug released was analyzed at 260 nm for dapsone, 307 nm for pralatrexate, 287 nm for trimethoprim, and 274 nm for KGN, and was determined as the mean value of triplicate determinations.

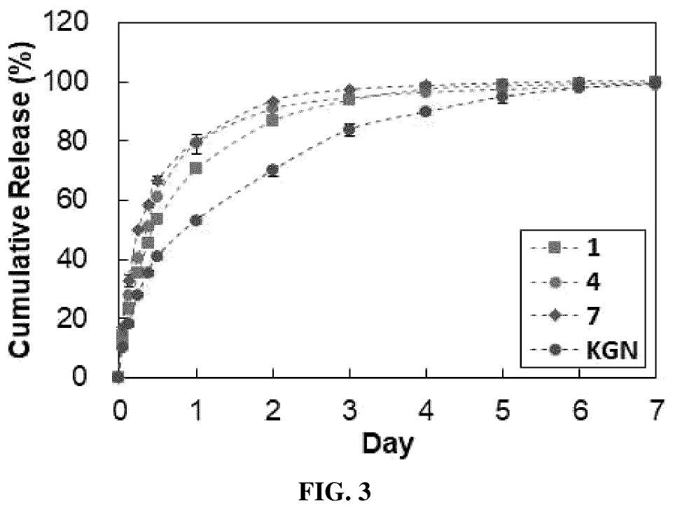

The results are shown in FIG. 3. 70% of dapsone (1) was released over 24 hours, while 79% of pralatrexate (4) and trimethoprim (7) were released over 24 hours. More than 95% of the antifolates (compounds 1, 4, and 7) were released over 3 days. Referring to the release profiles, the candidate antifolates were released from the PEG-PA thermogel during the first few days, indicating that each system can provide an antifolate pretreatment effect for TMSCs.

### Example 3. Evaluation of Chondrogenic Effects of Antifolates

### 3-1. Evaluation of Cytotoxicity of Antifolates to Stem Cells

Various antifolates were screened to evaluate their effects on cell proliferation and viability and their chondrogenic potential. The antifolates used were dapsone (1), methotrexate (2), pemetrexed (3), pralatrexate (4), proguanil (5), pyrimethamine (6), and trimethoprim (7). Stem cells loaded in a PEG-PA thermogel scaffold containing each antifolate or KGN were cultured in a DMEM growth medium containing FBS (10.0% v/v) for 21 days. A control experiment was performed under the same conditions, except that no antifolate was added.

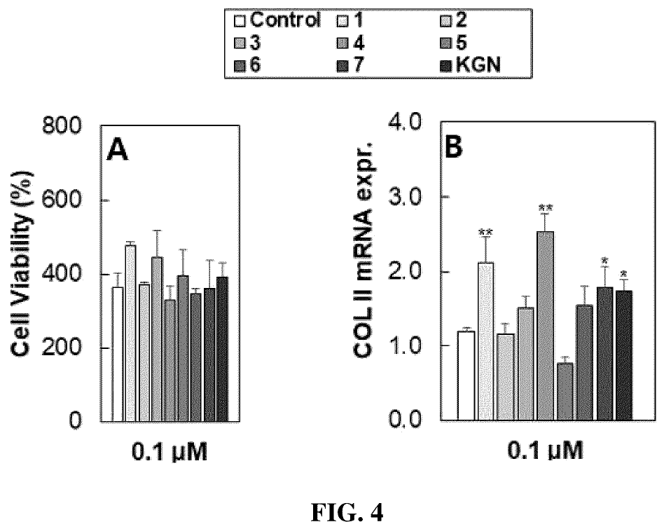

Cell proliferation was examined as a function of the antifolate concentration in the thermogel using a Cell Counting Kit-8 (CCK-8) (FIG. 4A). The cells on day 21 of culture were compared with those on day 0 to check whether the antifolate would significantly interfere with cell proliferation. Specifically, CCK-8 solution (10.0% v/v in DMEM containing 1.0% v/v PS) was added to each well (1.5 ml/well) of a plate, cells were cultured in each well for 3 hours at 37° C. under 5% CO₂, and then the absorbance at 450 nm was measured using a microplate reader (iMARK™, Bio-Rad, USA). The absorbance at 655 nm was used as a baseline. Cell viability was analyzed using a Live/Dead Kit (Molecular Probes, USA). The cell culture medium was replaced with PBS containing ethidium homodimer-1 (2.0 μM) and calcein acetoxy methyl ester (2.0 μM), followed by culture for 30 minutes. After 3D culture for 0, 14, and 21 days, the cells were observed under an Olympus IX71 fluorescence microscope and images thereof were acquired using the Olympus DP2-BSW software.

As a result, it was confirmed that all of the antifolates were not cytotoxic at a concentration of 0.1 μM.

### 3-2. Evaluation of Chondrogenic Effects of Antifolates

Expression of chondrogenic biomarkers was investigated in the presence of antifolates. The expression of COL II, the most widely used biomarker for chondrogenic differentiation of stem cells, was investigated at the mRNA level (FIG. 4B). At a concentration of 0.1 μM, COL II expression significantly increased (p<0.05) in dapsone (1), pralatrexate (4), trimethoprim (7), and KGN (positive control compound), and the mRNA expression level of COL II increased by 1.8-fold, 2.1-fold, 1.5-fold, and 1.5-fold in dapsone (1), pralatrexate (4), trimethoprim (7), and KGN, respectively, compared to that in the control. Thereamong, pralatrexate was confirmed to have a significantly better effect on COL II expression.

### Example 4. Stem Cell Culture in Thermogel and Evaluation of Chondrogenesis-Promoting Effect

### 4-1. 3D Stem Cell Culture in Thermogel

Based on the screening results obtained in Example 3, dapsone (1), pralatrexate (4), and trimethoprim (7) were selected as antifolate candidates. Then, detailed studies were performed on the selected antifolates at a concentration of 0.1 μM, which does not show significant toxicity to stem cells, and a KGN-containing system at 0.1 μM was used as a positive control.

Human TMSCs used as stem cells were donated from Ewha Womans University Mokdong Hospital (Seoul, Korea). The cells were isolated from a 6-year-old male donor after tonsillectomy in compliance with the ethical guidelines of the University. DMEM (Corning, USA) containing 10.0% (v/v) fetal bovine serum (FBS, Corning, USA), 1.0% (v/v) penicillin/streptomycin (PS, Corning, USA) and 1.0% (v/v) antibiotic-antimitotic (AA, Corning, USA) was used as a growth medium, and the isolated human TMSCs were cultured using the growth medium in traditional polystyrene culture dishes at 37° C. under 5% CO₂ atmosphere. Then, the stem cells (passage 7, 0.5×10⁶ cells/well) were mixed with the aqueous PEG-PA solution (10.0 wt % in growth medium) containing an antifolate or KGN at 20° C. A system containing no antifolate or KGN was used as a control. The TMSC-suspended solution was injected into a 24-well culture plate (0.2 ml/well) at 37° C. to form a hydrogel matrix. The medium was replaced every 3 days during a 3D culture period of 21 days, and each experiment was performed in triplicate.

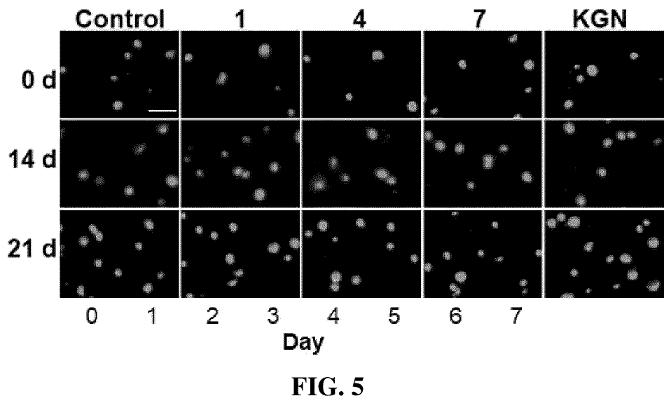

First, after 14 and 21 days of 3D cell culture using the PEG-PA thermogel, live and dead cells were stained in green and red, respectively, and images thereof were obtained using a Live/Dead assay kit (FIG. 5). In all of the images, most cells were found to be alive, and the cell density increased.

4-2. Evaluation of Chondrogenic Differentiation-Inducing Effect at mRAN Level

Chondrogenic differentiation of TMSCs was investigated at the mRNA and protein levels of chondrogenic biomarkers such as COL II, SOX 9, and ACAN. Gene expression levels were determined compared to that of a control system containing no antifolate using equal amounts of cDNA. COL I, an osteogenic biomarker, was also used to determine whether osteogenic differentiation occurred when an antifolate was used.

Specifically, after 0, 14, and 21 days of 3D culture, mRNA was extracted from the cells in the thermogel by using a TRIZOL™ reagent (Invitrogen, USA) according to the manufacturer's protocol, and the mRNA concentration was assayed by a Nano Drop 2000 spectrometer (Thermo Scientific, USA). Equal amounts of cDNA were used in qPCR to determine gene expression levels compared to that of the control system containing no antifolate. Then, total cDNA was synthesized using a ReverTra Ace® qPCR RT kit (Toyobo, Japan), and mRNA expression levels were measured using a CFX96™ real-time RT-PCR system (Bio-Rad, USA) and an iQ™ SYBR® Green Supermix solution (Bio-Rad, USA). The relative expression levels of target genes were calculated as 2^{-ΔΔCt} by the following calculation equation: ΔΔCt=(gene A−GAPDH)ₜ−(gene A−GAPDH)₀. Here, the subscript 0 refers to data on day 0 (3 hours). The primer sequences used for amplification are shown in Table 1 below, and each test value was determined as the mean value of triplicate determinations.

TABLE 1

| | | Annealing |
|---|---|---|
| Genes | Primer sequences | Temp. (° C.) |
| COL II | F: 5′-AGG AGG CTG GCA GCT GTG TGC-3′ | 65.0 |
| | R: 5′-CAC TGG CAG TGG CGA GGT CAG-3′ | 62.8 |
| SOX 9 | F: 5′-GCA GCG AAA TCA ACG AGA AAC-3′ | 55.0 |
| | R: 5′-TCC AAA CAG GCA GAG AGA TTT AG-3′ | 54.5 |
| ACAN | F: 5′-CAG AGC AAG ACT CTG TCT CAA A-3′ | 54.5 |
| | R: 5′-GGG ACA AGA GCC TCA TCA AA-3′ | 54.8 |
| COL I | F: 5′-CTC CTC AAG GGC TCC AAC G-3′ | 58.0 |
| | R: 5′-CAT CGA CAG TGA CGC TGT AGG-3′ | 57.5 |
| GAPDH | F: 5′-CTC CTC ACA GTT GCC ATG TA-3′ | 54.5 |
| | R: 5′-GTT GAG CAC AGG GTA CTT TAT TG-3′ | 53.9 |

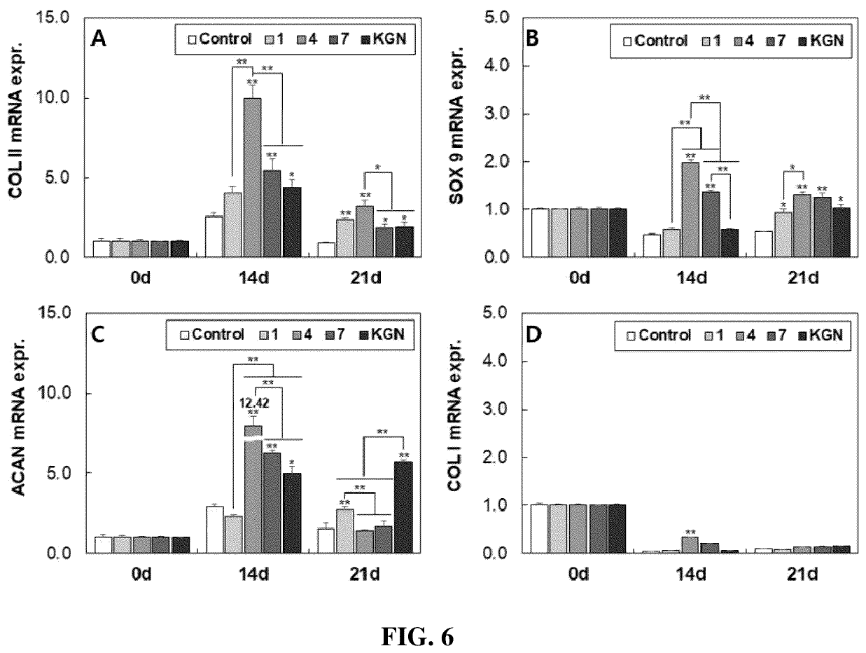

In Table 1 above, COL II, SOX 9, ACAN, COL I, and GAPDH denote type II collagen, SRY box transcription factor 9, aggrecan, type I collagen, and glyceraldehyde-3-phosphate-dehydrogenase, respectively. F and R denote forward and reverse primers, respectively. As a result, COL II expression was the highest in the system containing pralatrexate (4), increased by 10-fold on day 14 compared to day 0 (3 hours after the start of culture), and was 2-fold higher than that in KGN. As of day 14, COL II mRNA levels increased by 3.9-, 2.1- and 1.7-fold in the systems containing pralatrexate, trimethoprim and KGN, respectively, compared to that in the control system containing no antifolate (FIG. 6A). COL II expression levels increased by 2.6-fold, 3.5-fold, 2.1-fold and 2.1-fold in the systems containing dapsone, pralatrexate, trimethoprim and KGN, respectively, compared to that in the control system. Similarly, SOX 9 expression significantly (p<0.01) increased in the systems containing pralatrexate and trimethoprim on days 4 and 21 (FIG. 6B). In particular, the system containing pralatrexate exhibited the highest SOX 9 mRNA level.

On day 14, the systems containing pralatrexate and trimethoprim increased SOX 9 mRNA expression levels by 4.2-fold and 2.9-fold, respectively, compared to the control system. ACAN mRNA expression levels also showed a tendency similar to SOX 9 mRNA expression levels. ACAN mRNA levels on day 14 increased by 12-, 6-, and 5-fold for the systems containing pralatrexate, trimethoprim, and KGN, respectively, compared to those on day 0 (3 hours after the start of culture) (FIG. 6C). ACAN expression levels on day 14 increased by 4.3-fold, 2.2-fold, and 1.7-fold in the systems containing pralatrexate, trimethoprim, and KGN, respectively, compared to that in the control system containing no antifolate. On day 20, the system containing KGN exhibited the highest ACAN mRNA levels, which is approximately 3.7 times that in the control system. On the other hand, expression of the osteogenic differentiation biomarker COL I was significantly lower than that on day 0 under all conditions (FIG. 6D). These results indicate that the antifolates induce stem cells to differentiate into chondrocytes and inhibit differentiation of stem cells into osteocytes.

### 4-2. Evaluation of Chondrogenic Differentiation-Inducing Effect at Protein Level

Protein expression for COL II, SOX 9, ACAN and COL I was investigated using immunofluorescence assay for stem cells cultured for 14 days. Specifically, TMSCs, which were 3D cultured with antifolates or KGN for 14 days and then encapsulated in thermogel scaffolds, were fixed with 4.0% paraformaldehyde (PFA) for 12 hours and quickly frozen at −70° C. Then, the thermogels were cryosectioned to a thickness of 10 μm using CM1850 (Leica, Germany). All the samples were treated with Triton (0.25% v/v in PBS), and then blocked with bovine serum albumin (1.0% w/v in PBS), and treated with anti-COL II, anti-SOX 9, anti-ACAN, and anti-COL I primary antibodies. Next, the samples were washed with PBS and treated with the following secondary antibodies: goat anti-mouse IgG H&L Alexa Fluor®594 for COL II and ACAN, and goat anti-rabbit IgG H&L Alexa Fluor®594 for SOX 9 and COL I. All the antibodies were purchased from Abcam (USA) and tested according to the manufacturer's protocol. In addition, each sample was stained for nuclei and F-actin with DAPI (Molecular probes, USA) and phalloidin (PHD) (Abcam, USA), respectively. Images were obtained using an Olympus IX71 fluorescence microscope and Olympus DP2-BSW software.

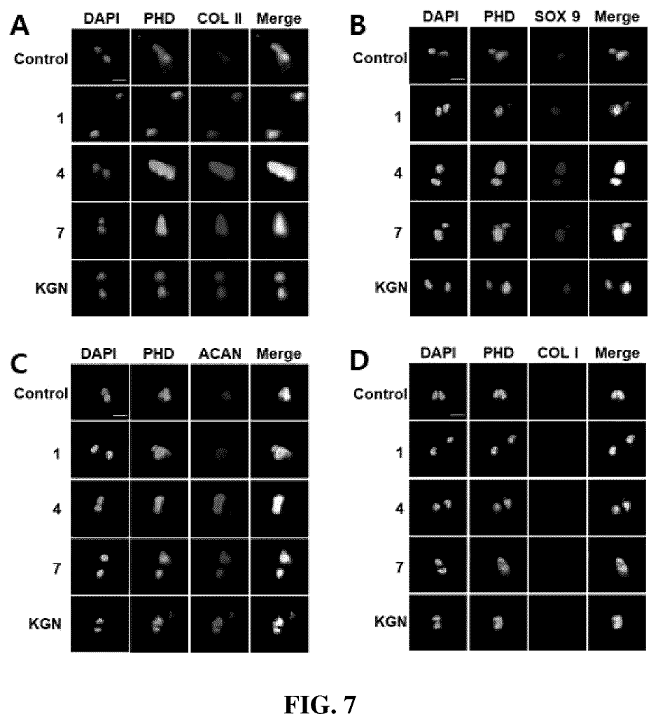

The results are shown in FIG. 7. For immunofluorescence assay, the cell nucleus and actin were stained blue and green with DAPI and PHD, respectively, and the antibodies for each protein were stained red. The fluorescence intensity (red) of each of COL II, SOX 9, and ACAN was highest in the system containing pralatrexate, while the fluorescence intensity of COL I was significantly lower under all conditions. These results were consistent with the mRNA expression results.

### Example 5. Evaluation of Effect of Antifolates on Regulation of Chondrogenic Differentiation-Related Factor

Filamin A is an actin-binding protein that regulates the cytoskeletal network involved in chondrogenic differentiation. When KGN binds to filamin A in the cytosol, the interaction between filamin A and CBFβ is broken, and when the dissociated CBFβ enters the nucleus, CBFβ regulates RUNX1, thereby promoting differentiation of MSCs into chondrocytes. Whether the antifolate can be involved in this mechanism and regulate chondrogenic differentiation-related factors was examined by Western blot analysis and immunofluorescence assay.

Specifically, for Western blot analysis, stem cells (TMSCs) were starved overnight and then treated with each antifolate or KGN in serum-free growth medium. Then, cytosolic and nuclear proteins of the TMSCs were obtained using a nuclear/cytosolic fractionation kit (Cell Biolabs Inc., USA) and total protein was obtained using a cell extraction buffer (Invitrogen, USA) containing a protease inhibitor and phenyl methane sulfonyl fluoride (1.0 mM). Each of the cytosolic and nuclear proteins was loaded, detected with anti-CBFβ primary antibody (Cell Signaling Technology, USA), and then treated with anti-rabbit IgG HRP-conjugated secondary antibody (Cell Signaling Technology, USA) and incubated. Then, the proteins were visualized with an enhanced chemiluminescence substrate (Bio-rad, USA) using an Amersham Imager 600 (GE, UK).

For immunofluorescence assay, stem cells were starved overnight and then treated with each antifolate or KGN in serum-free growth medium. Each cell was treated with bovine serum albumin (1.0% w/v in PBS), and then treated with anti-CBFβ primary antibody (Abcam, USA) and anti-RUNX1 primary antibody (Santa Cruz Biotechnology, USA) and incubated. The cells were treated with anti-rabbit IgG H&L secondary antibodies (Abcam, USA) and anti-mouse IgG H&L secondary antibodies (Abcam, USA) against CBFβ and RUNX1, respectively. The cell nuclei were stained with DAPI (Molecular probes, USA), and each image was obtained using an Olympus IX71 fluorescence microscope and Olympus DP2-BSW software.

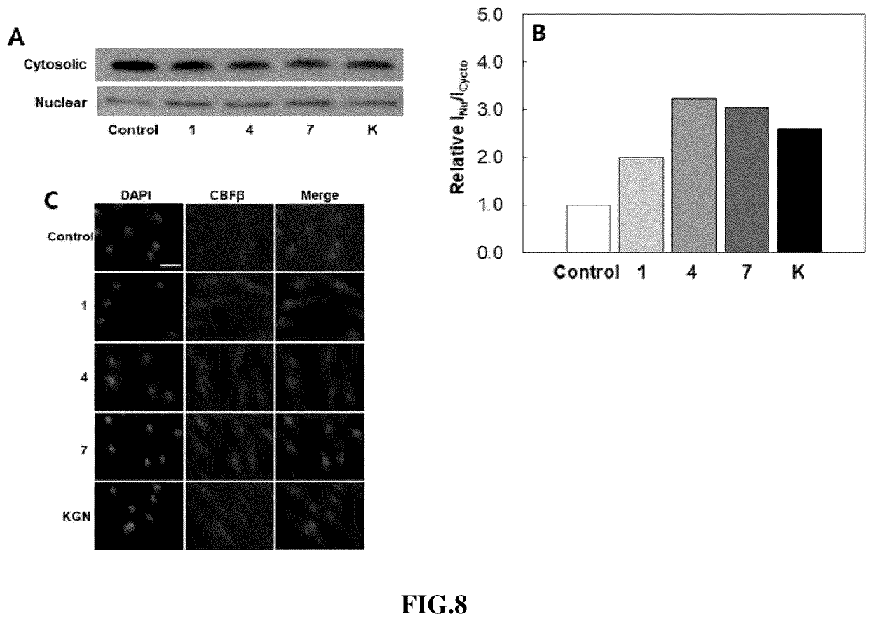

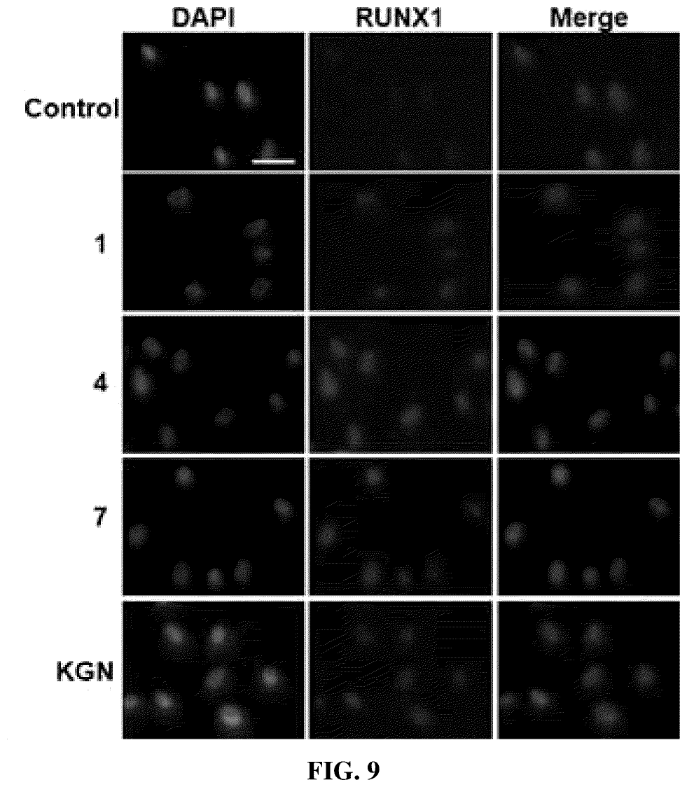

The results are shown in FIGS. 8 and 9. In Western blot analysis, it was confirmed that CBFβ in the cytosol decreased along with an increase in CBFβ in the nucleus, and that when the stem cells were treated with the antifolate, the nuclear translocation of CBFβ increased (FIG. 8A). As a result of comparing the nuclear band intensity versus the cytosolic band intensity, it was confirmed that treatment with pralatrexate increased the nuclear translocation of CBFβ (FIG. 8B). Immunofluorescence images also show that the expression of CBFβ in the nucleus was increased by antifolate treatment (FIG. 8C).

RUNX1 expression was investigated by immunofluorescence (FIG. 9). Treatment with pralatrexate increased the fluorescence intensity of RUNX1 in the nucleus compared to the control. It was confirmed that the antifolate promoted chondrogenic differentiation of stem cells by increasing the nuclear translocation of CBFβ, which upregulates RUNX1-mediated chondrogenesis of stem cells.

Statistical significance between all result values was analyzed by one-way ANOVA with the Tukey test. * and ** represent p<0.05 and p<0.01, respectively.

### INDUSTRIAL APPLICABILITY

The present invention relates to a composition for inducing differentiation of stem cells into chondrocytes containing an antifolate and a method of treating cartilage injury disease using the same, and is capable of differentiating stem cells into chondrocytes economically, stably, and efficiently by using a clinically approved antifolate such as dapsone, pralatrexate, trimethoprim, or a combination thereof.

1. A composition for inducing differentiation of stem cells into chondrocytes, containing an antifolate.

2. The composition according to claim 1, wherein the stem cells are selected from the group consisting of adipose-derived mesenchymal stem cells, bone marrow-derived mesenchymal stem cells, umbilical cord blood-derived stem cells, embryonic stem cells, tonsil-derived mesenchymal stem cells, and synovial mesenchymal stem cells.

3. The composition according to claim 1, wherein the antifolate is dapsone, pralatrexate, trimethoprim, or a combination thereof.

4. The composition according to claim 1, wherein the antifolate is pralatrexate.

5. The composition according to claim 1, wherein a concentration of the antifolate is 0.05 μM to 100 μM.

6. A pharmaceutical composition for treating cartilage injury disease, containing an antifolate and stem cells.

7. The pharmaceutical composition according to claim 6, wherein the cartilage injury disease is arthritis, cartilage injury, cartilage defect, degenerative arthritis, rheumatoid arthritis, fracture, plantar fasciitis, lateral humerus epicondylitis, osteomalacia, or a combination thereof.

8. A sustained-release dosage form for inducing differentiation of stem cells into chondrocytes, comprising the composition according to claim 1.

9. The sustained-release dosage form according to claim 8, wherein the sustained-release dosage form is a thermogel.

10. The sustained-release dosage form according to claim 8, wherein the sustained-release dosage form comprises a poly(ethylene glycol)-poly(L-alanine) copolymer.

11. The sustained-release dosage form according to claim 8, wherein the sustained-release dosage form further comprises stem cells.
